(19) United States
(12) Patent Application Publication
(10) Pub. No.: US 2020/0188433 A1
(43) Pub. Date: Jun. 18, 2020

(54) aNK AND IL-12 COMPOSITIONS AND METHODS

(71) Applicant: NANTCELL, INC., Culver City, CA (US)

(72) Inventors: Patrick SOON-SHIONG, Culver City, CA (US); Kayvan NIAZI, Culver City, CA (US)

(21) Appl. No.: 16/498,315

(22) PCT Filed: Mar. 26, 2018
(86) PCT No.: PCT/US2018/024285

§ 371 Date: Sep. 26, 2019

Related U.S. Application Data
(60) Provisional application No. 62/477,232, filed on Mar. 27, 2017.

(51) Int. Cl. A61K 35/17; C07K 14/54; C07K 14/55; C12N 15/86

(52) U.S. Cl. CPC A61K 35/17; C07K 14/5434; C12N 2710/10343; C12N 15/86; C07K 14/55

(57) ABSTRACT

Contemplated treatment compositions and methods are directed to co-administration of sensitized genetically modified NK cells and recombinant IL-12, wherein the genetically modified NK cells were preferably sensitized by constitutive exposure to IL-2 and wherein the IL-12 is expressed from a recombinant virus or given as an IL-12-antibody conjugate. Such treatment increases IFNγ secretion by the sensitized NK cells, and advantageously also increases expression of NKG2D.

This application claims priority to our copending US provisional application with the Ser. No. 62/477,232, which was filed Mar. 27, 2017.

### FIELD OF THE INVENTION

The field of the invention is cancer treatments and methods using natural killer cells and immune stimulatory cytokines, and especially activated NK cells and IL-12.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

More recently, cell-based cancer treatments with genetically modified NK cells have gained attention due to positive treatment outcomes, particularly where NK92 derivatives such as activated NK cells (aNK cells), genetically modified NK cells with high affinity CD16 receptors (haNK cells), or chimeric antigen receptors (taNK cells) were used. While such cell-based treatments are conceptually attractive, the tumor microenvironment and other patient-specific factors will often reduce their cytotoxic activity, and various attempts have been made to modulate cytotoxicity in NK92 cells.

Interleukin-2 (IL-2) and interleukin-12 (IL-12) are cytokines that are known to elicit strong antitumor effects by stimulating unmodified immune cells, including T cells and natural killer (NK) cells. Although either cytokine stimulates the proliferation of T cells, the production of interferon-γ (IFN-γ) by NK cells, and ultimately the cytolytic activity, the magnitude, and the spectrum of stimulatory effects by IL-2 and IL-12 are different (see e.g., J. Leukoc. Biol. 58: 225-233; 1995). Although IL-2 is a stronger stimulator of proliferation and cytolytic activity, IL-12 is a stronger inducer of IFN-γ from unmodified NK cells and activated T cells. IFN-γ mRNA was shown to have increased stability in NK cells co-stimulated with IL-2 and IL-12 (see e.g., Molecular And Cellular Biology, March 2002, p. 1742-1753). However, the IL-2 and IL-12 concentrations used in vitro may not necessarily reflect achievable or even desirable levels in vivo. Indeed, IL-2 systemic administration of IL-2 is associated with relatively high toxicity and capillary leak syndrome, while several deaths have been attributed to administration of IL-12. Moreover, the response of primary NK cells to various cytokines is not necessarily the same as the response of NK92 cells, which are NK cell tumor cells.

Therefore, there remains a need for compositions and methods to treat cancer using cell based therapeutics, especially NK cell based therapeutics where NK cells are stimulated in a clinically safe manner.

### SUMMARY OF THE INVENTION

The inventive subject matter is directed to various compositions and methods of NK cell activation, and particularly activation of aNK cells and other genetically modified NK92 derivatives by constitutive exposure to IL-2 and further exposure IL-12, which is preferably expressed in vivo or antibody conjugated. Thusly preconditioned cells exhibited substantial IFN-γ secretion and avoided systemic toxicities that would otherwise be encountered by in vivo administration of IL-2 and IL-12 to a patient receiving aNK cells. Moreover, so activated NK cells had increased NKG2D expression, which further enhanced innate cytotoxicity.

In one aspect of the inventive subject matter, the inventors contemplate a method of stimulating a genetically modified NK cell that includes one step of exposing a genetically modified NK cell constitutively to IL-2 to thereby sensitize the genetically modified NK cell to IL-12, and another step of exposing the sensitized cell to IL-12 to stimulate interferon gamma (IFNγ) secretion by the sensitized cell. Most typically, the step of exposing the sensitized cell to IL-12 also increases expression of NKG2D.

In some embodiments, the genetically modified NK cell is an aNK cell, and the genetically modified NK cell is constitutively exposed to IL-2 at a concentration of at least 100, or at least 200, or at least 500, or at least 1,000 IU/ml. Furthermore, it is contemplated that the IL-2 may be a pegylated IL-2. Alternatively, the genetically modified NK cell may also be constitutively exposed to IL-2 by intracellular expression of IL-2. Thus, contemplated genetically modified NK cell also include a haNK cell.

The IL-12 may be expressed from a cell that is infected with a recombinant virus, wherein the recombinant virus includes a sequence segment that encodes the IL-12. In such case, it is also contemplated that the recombinant virus includes a second sequence segment that encodes at least one of a tumor and patient specific antigen, a tumor associated antigen, and a tumor specific antigen. Alternatively, or additionally, the IL-12 may be coupled to an antibody, which preferably binds to a cancer cell. It is still further contemplated that the genetically modified NK cell is exposed to the IL-2 in vitro, and that the sensitized cell is administered to a patient, and/or that the sensitized cell is exposed to the IL-12 in vitro, and that the sensitized cell is administered to a patient.

Therefore, in another aspect of the inventive subject matter, the inventors also contemplate a method of treating cancer that includes a step of administering a sensitized genetically modified NK cell to an individual diagnosed with cancer, wherein the genetically modified NK cell is sensitized by constitutive exposure to IL-2; and another step of administering an IL-12 antibody conjugate or a recombinant virus to the individual that encodes IL-12 to stimulate interferon gamma (IFNγ) secretion by the sensitized genetically modified NK cell. Typically, wherein the IL-12 antibody or the IL-12 expressed from the recombinant virus will also increase the expression of NKG2D. With respect to the NK cells, the IL-2, the IL-12, and methods of administration, the same considerations as noted above apply.

Therefore, and viewed from another perspective, the inventors also contemplate a sensitized genetically modified NK cell for use in immune therapy of cancer, wherein the genetically modified NK cell is sensitized by constitutive exposure to IL-2, and wherein the immune therapy uses a recombinant virus that expresses IL-12 or an IL-12 antibody conjugate. Most typically, such cell is an aNK cell, which may be sensitized by constitutive exposure to at least 100 IU/ml IL-2, by pegylated IL-2, or by intracellular expression of IL-2. Therefore, suitable genetically modified NK cells also include haNK cells. Suitable recombinant viruses may further include a sequence segment that encodes at least one of a tumor and patient specific antigen, a tumor associated antigen, and a tumor specific antigen, and/or the immune therapy may use the IL-12 antibody conjugate where the antibody preferably binds to a cancer cell.

In yet another aspect of the inventive subject matter, the inventors also contemplate a method of increasing activity of NK cells or CD8⁺ T-cells in a mammal. Preferred methods will include a step of infecting cells of the mammal with a plurality of recombinant viral particles, each viral particle comprising a recombinant nucleic acid segment encoding IL-12 operably coupled to a promoter sequence that drives expression of IL-12 in a cell infected with the recombinant viral particles. Most typically, the plurality of recombinant viral particles is sufficient to cause expression of a quantity of IL-12 in the infected cells that increases expression of NKG2D on the NK cells or CD8⁺ T-cells in the mammal infected with the virus (typically with at least 10¹⁰ or 10¹¹ viral particles. While not limiting to the inventive subject matter, it is typically preferred that the recombinant viral particles are genetically modified adenovirus Ad5 [E1-, E2b-] particles. Where desired, the step of infecting the cells may be performed in vitro, and the infected cells are then administered to the patient. It is contemplated that suitable NK cells include allogenic NK92 derivative cells (e.g., aNK cells, haNK cells, taNK cells). Moreover, it is contemplated that such methods may further include a step of administering to the patient a pharmaceutical agent (e.g., IL-15, IL-2, doxorubicin, or a gluten peptide fragment) that increases NKG2D-based cytotoxicity of NK cells and T-cells.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIGS. 1A and 1B are exemplary graphs showing lack of IFNγ secretion of aNK cells in response to IL-12 stimulation without prior constitutive exposure to IL-2.

FIGS. 2A and 2B are exemplary graphs showing high IFNγ secretion of haNK cells in response to IL-12 stimulation with prior constitutive exposure to IL-2.

FIGS. 3A and 3B are exemplary graphs showing high IFNγ secretion of aNK/haNK cells in response to IL-12 stimulation without prior constitutive exposure to IL-2 (3A) and with constitutive exposure to IL-2 (3B).

FIG. 4 is an exemplary photograph of a SDS-PAGE showing IL-12 expression in various samples.

FIGS. 5A and 5B are graphs depicting weight (5A) and body temperature (5B) of non-human primates (NHP) infected with Ad5[E1-,E2b-]-IL-12.

FIG. 6 is a graph depicting serum IL-12 levels of non-human primates infected with Ad5 [E1-,E2b-]-IL-12.

### DETAILED DESCRIPTION

The inventors have now discovered that NK cells, and especially genetically modified NK92 cells can be effectively stimulated to secrete IFNγ in response to IL-12 exposure, and as such are able to promote cytotoxic and NK cell activity, increase antigen presentation on antigen presenting cells via increased MHC-I/II expression, and bias an immune response towards a Th1 response. Notably, and as discussed in more detail below, various NK92 cells are already propagated in a culture medium containing IL-2, which is renewed periodically, typically every two to three days (depending on cell density). However, these cells fail to be responsive to IL-12. Unexpectedly, when IL-2 is supplied to the same cells in a constitutive or continuous manner, the cells are sensitive to IL-12 signaling, produce significant quantities of IFNγ and have increased cytotoxicity.

The terms ‘constitutive exposure’ or ‘constitutively exposing’ in conjunction with IL-2 as used herein means that biologically active IL-2 is supplied or produced in a continuous (or semi-continuous) manner such that variations in biologically active IL-2 concentration at or in the cell (or IL-2 stimulation) will vary by no more than 20% over 48 hours. Therefore, in some embodiments, variations in biologically active IL-2 concentration at or in the cell (or IL-2 stimulation) will vary by no more than 15% over 48 hours, or no more than 10% over 48 hours, or no more than 7% over 48 hours, or no more than 5% over 48 hours, or no more than 1% over 48 hours.

Constitutive exposure to IL-2 is believed to more closely resemble natural exposure to IL-2, leading to durable sensitivity to IL-12 and IFNγ secretion triggered by exposure to IL-12. In contrast, exposure of NK-92 cells to IL-2 in an intermittent fashion as is the case in customary NK-92 cell culture where culture media are renewed every two to three days may lead to inactivation and/or degradation of biologically active IL-2, possibly due to binding by serum proteins or proteolysis by serum proteases that are present in the serum components (typically horse serum and fetal bovine serum) of the culture media. Consequently, and without wishing to be bound by any theory or hypothesis, the inventors believe that customary NK-92 cell culture conditions promote intermittent or ‘pulsed’ IL-2 signaling which supports growth of NK-92 cells but fails to support cell signaling to render the NK-92 cells sensitive to IL-12 and IL-12 dependent IFNγ secretion as well as increased (as compared to non-IL-12 stimulated cells) expression of NKG2D.

Based on these findings as shown in more detail below, the inventors contemplate that NK-92 cells and derivatives thereof can be sensitized to IL-12 and IL-12 dependent IFNγ secretion and increased expression of NKG2D by constitutively exposing the cells to IL-2. For example, in one embodiment constitutive exposure can be performed by continuous or semi-continuous addition of IL-2 to a culture medium to thereby maintain the concentration of biologically active IL-2 substantially constant. Therefore, it is contemplated that the concentration of biologically active IL-2 in the medium varies over 48 hours by no more than 15%, or by no more than 10%, or by no more than 7%, or by no more than 5%, or by no more than 3%. As will be readily appreciated, biological activity of IL-2 can be quantified using known procedures, e.g., using a CTLL-2 cell proliferation assay (J Immunol Methods. 2009 Aug. 31; 348(1-2): 83-94).

Continuous or semi-continuous addition of IL-2 can be done in numerous manners, including use of a peristaltic pump or metered injector. Alternatively, and in less preferred aspects, continuous or semi-continuous addition of IL-2 can be done by media renewal in a frequent fashion (e.g., every two hours, every four hours, every eight hours, etc.). Where multiple or continuous additions are not preferred, the inventors also contemplate that the constitutive exposure can also be achieved using formulations that release IL-2 in a relatively slow manner. For example, delayed release of IL-2 (or increased stability against protein binding and/or protease digest) can be done by pegylation of IL-2 as is known from NKTR-214 (Nektar Therapeutics; 455 Mission Bay Blvd South; San Francisco, Calif. 94158). Here, pegylated IL-2 is believed to be a prodrug form of biologically active IL-2 that releases PEG chains over time to produce biologically active IL-2 (PLoS One. 2017 Jul. 5; 12(7):e0179431). Advantageously, such pegylated IL-2 can be systemically administered and as such allows for constitutive exposure of NK/NK92 cells and their derivatives to IL-2 in vivo while at the same time systemic side effects of IL-2 are reduced, or even entirely avoided.

Alternatively, or additionally, it should be noted that constitutive exposure can also be achieved using antibody conjugated IL-2 as such conjugates have shown increased stability, presumably due to decreased binding to serum proteins and decreased proteolysis by serum proteases. Once more, such antibody-drug conjugates will advantageously be administrable to a patient in vivo. In this embodiment, however (and in contrast to NKTR-214), delivery of IL-2 and with that activation of NK cells, is possible with high specificity and selectivity as far as location is concerned. For example, such antibody-drug conjugates may target tumor markers that are patient and tumor specific (i.e., tumor neoepitopes), cancer associated, cancer specific, or specific to necrotic tissue commonly found in a tumor microenvironment. Of course, it should be appreciated that the antibody portion in such antibody-drug conjugates may be a full IgG antibody, or any suitable fragment thereof (e.g., scFv, Fab, Fab′, F(ab′)₂, etc.).

As will be readily appreciated, such antibody-drug conjugates may be prepared by chemical conjugation using cleavable (e.g., via disulfide bond or hydrozone, or proteolytic site, etc.) or non-cleavable linkers (e.g., via maleimide-modified PEG). Alternatively, the conjugation may also be done using recombinant cloning in which the N- or C-terminus of the heavy or light chain (or fragment thereof) is modified to also encode in frame a linker portion and IL-2. Thus, chimeric recombinant proteins can be prepared that have an antibody portion that preferably binds to a component of a tumor cell, a linker, and an IL-2 portion. Notably, exemplary antibody-drug conjugates with IL-2 retained significant activity as is shown in more detail below.

Regardless of the particular form of IL-2 it is generally contemplated that constitutive exposure of NK/NK92 cells and their derivatives to IL-2 (and modified forms of IL-2) will be at a concentration of between about 10-50 IU/ml, or between about 50-150 IU/ml, or between about 150-300 IU/ml, or between about 300-500 IU/ml, or between about 500-1,000 IU/ml, or even higher (as determined by CTLL-2 proliferation assay). Moreover, it is generally contemplated that the IL-2 concentration remains substantially constant over at least a limited period of time. For example, it is typically preferred that the concentration of the biologically active IL-2 fluctuates less than 25%, or less than 20%, or less than 15%, or less than 10%, or less than 7%, or less than 5%, or less than 3% as measured in % change of IU/ml over a period of 72 hours, or over a period of 60 hours, or over a period of 48 hours, or over a period of 36 hours, or over a period of 24 hours, or over a period of 18 hours. Thus, viewed from a different perspective, suitable concentration of the biologically active IL-2 will be maintained throughout the entire cell culture between 50-70 IU/ml, or between 70-100 IU/ml, or between 100-120 IU/ml, or between 120-150 IU/ml, or between 150-200 IU/ml, or between 200-230 IU/ml, or between 230-250 IU/ml, or between 250-280 IU/ml, or higher.

In yet further contemplated aspects, constitutive exposure of NK/NK92 cells and their derivatives to IL-2 can also be achieved by intracellular expression of recombinant IL-2 in the respective cells. Most preferably, intracellular expression is driven from a constitutively active promoter to achieve constant expression levels, and is expressed in a form that is not secreted (i.e., lacks export signal sequence, and may include an endoplasmic or cytoplasmic retention sequence). Such intracellular expression is believed to provide the same functional impact to the cell as constitutive exposure to externally provided IL-2. Indeed, as is shown in more detail below, the inventors discovered that where NK92 cells or derivatives were genetically engineered to express and intracellularly retain IL-2, the cells were unexpectedly sensitized to IL12 stimulation as measured by IFNγ secretion and/or increased expression of NKG2D. As will be readily appreciated, recombinant expression and intracellular retention of IL-2 can be done in numerous manners, and all of such methods are deemed suitable for use herein (see e.g., Oncotarget 2016 Dec. 27; 7(52): 86359-86373). Among other benefits, it should be noted that such recombinant cells can be administered to a patient in vivo without the need to administer to the same patient IL-2. Such modified cells will be sensitized to IL-12 to secrete IFNγ upon IL-12 stimulation. Indeed, sensitization by constitutive exposure (external or internal) to IL-2 provided substantial quantities of IFNγ that is thought to provide a therapeutic effect in the context of concomitant immune therapy, particularly as NKG2D expression in such stimulated cells was also significantly increased.

With respect to NK cells it is contemplated that all NK cells are deemed suitable for use herein and thus include autologous NK cells from a patient (e.g., isolated from whole blood, or cultivated from precursor or stem cells using methods known in the art), and various allogenic NK cells. However, in preferred aspects of the inventive subject matter, the NK cells are genetically engineered to achieve one or more desirable traits, and particularly include NK-92 cells and derivatives thereof. For example, suitable genetically engineered NK cell include NK-92 derivatives that are modified to have reduced or abolished expression of at least one killer cell immunoglobulin-like receptor (KIR), which will render such cells constitutively activated (via lack of or reduced inhibition).

NK-92 cells exhibit an unusual receptor expression profile, expressing a relatively large number of activating (e.g., NKp30, NKp46, 2B4, NKGD, CD28) receptors. Conversely, NK-92 cells also express few inhibitory receptors (e.g., NKGA/B, low levels of KIR2DL4, ILT-2), and lack most of the killer inhibitory receptors (KIRs) clonally expressed on normal NK cells. In addition, NK-92 expresses relatively high levels of molecules involved in the perforin-granzyme cytolytic pathway as well as additional cytotoxic effector molecules including tumor necrosis factor (TNF)-superfamily members FasL, TRAIL, TWEAK, TNF-alpha, indicating the ability to kill via alternative mechanisms. Moreover, NK-92 cells also express other molecules implicated immune effector cell regulation (CD80, CD86, CD40L, TRANCE) whose relevance in NK killing is unclear.

Moreover, suitable NK cells may have one or more modified MR that are mutated such as to reduce or abolish interaction with MHC class I molecules. Of course, it should be noted that one or more KIRs may also be deleted or expression may be suppressed (e.g., via miRNA, siRNA, etc.). Most typically, more than one KIR will be mutated, deleted, or silenced, and especially contemplated MR include those with two or three domains, with short or long cytoplasmic tail. Viewed from a different perspective, modified, silenced, or deleted KIRs will include KIR2DL1, KIR2DL2, KIR2DL3, KIR2DL4, KIR2DL5A, KIR2DL5B, KIR2DS1, KIR2DS2, KIR2DS3, KIR2DS4, KIR2DS5, KIR3DL1, KIR3DL2, KIR3DL3, and/or KIR3DS1. Such modified cells may be prepared using protocols well known in the art. Alternatively, such cells may also be commercially obtained from NantKwest (see URL www.nantkwest.com) as aNK cells (‘activated natural killer cells).

In another preferred aspect of the inventive subject matter, the genetically engineered NK cells may also be NK-92 derivatives that are modified to express the high-affinity Fcγ receptor (CD16). Sequences for high-affinity variants of the Fcγ receptor are well known in the art (see e.g., Blood 2009 113:3716-3725), and all manners of generating and expression are deemed suitable for use herein. Expression of such receptor is believed to allow specific targeting of tumor cells using antibodies that are specific to a patient's tumor cells (e.g., neoepitopes), a particular tumor type (e.g., her2neu, PSA, PSMA, etc.), or that are associated with cancer (e.g., CEA-CAM). Advantageously, such antibodies are commercially available and can be used in conjunction with the cells (e.g., bound to the Fcγ receptor). Alternatively, such cells may also be commercially obtained from NantKwest as haNK cells (‘high-affinity natural killer cells).

In yet a further aspect of the inventive subject matter, the genetically engineered NK cell may also be genetically engineered to express a chimeric T-cell receptor. In especially preferred aspects, the chimeric T-cell receptor will have a scFv portion or other ectodomain with binding specificity against a tumor associated antigen, a tumor specific antigen, and a cancer neoepitope. As noted before, there are numerous manners of genetically engineering an NK cell to express such chimeric T-cell receptor, and all manners are deemed suitable for use herein. Alternatively, such cells may also be commercially obtained from NantKwest as taNK cells (‘target-activated natural killer cells’).

Where the cells are engineered to have affinity towards a cancer associated antigen or antibody with specificity towards a cancer associated antigen (e.g., via expression of a CAR), it is contemplated that all known cancer associated antigens are considered appropriate for use. For example, cancer associated antigens include CEA, MUC-1, CYPB1, etc. Likewise, where the cells are engineered to have affinity towards a cancer specific antigen or antibody with specificity towards a cancer specific antigen, it is contemplated that all known cancer specific antigens are considered appropriate for use. For example, cancer specific antigens include PSA, Her-2, PSA, brachyury, etc. Where the cells are engineered to have affinity towards a cancer neoepitope or antibody with specificity towards a cancer neoepitope, it is contemplated that all known manners of identifying neoepitopes will lead to suitable targets. For example, neoepitopes may be identified from a patient tumor in a first step by whole genome analysis of a tumor biopsy (or lymph biopsy or biopsy of a metastatic site) and matched normal tissue (i.e., non-diseased tissue from the same patient) via synchronous comparison of the so obtained omics information. So identified neoepitopes can then be further filtered for a match to the patient's HLA type to increase likelihood of antigen presentation of the neoepitope. Most preferably, such matching can be done in silico. In addition, all NK cells contemplated herein may also be genetically modified to express non-secreted IL-2 (e.g., retained in the ER compartment).

With respect to IL-12 it is generally contemplated that the IL-12 can be administered parenterally and systemically using protocols well known in the art. Moreover, IL-12 can also be specifically administered to a particular site in the body as an antibody-drug conjugate as already described for IL-2 antibody conjugates above. Advantageously, such administration to a specific site will focus IFNγ secretion of stimulated NK cells to a location where immune stimuli are desired (e.g., cancer or necrotic tissue, tumor microenvironment, etc.). However, in still further contemplated aspects it, it is also contemplated that the IL-12 is expressed from a recombinant expression system that can be transfected into autologous patient cells or allogenic immune competent cells (e.g., NK cells, NK92 derivatives, CD8⁺ and/or CD4⁺ T-cells, dendritic cells, macrophages, etc.) as is further described in more detail below. Such recombinant system may also include one or more sequence portions that encode proteins other than IL-12, and especially one or more tumor or cancer specific antigens, and/or co-stimulatory molecules, and/or checkpoint inhibitors. Advantageously, it should be noted that where immune competent cells produce recombinant IL-12, stimulated NK cells interacting with the immune competent cells may provide further activation to the immune competent cells via IFNγ secretion.

For example, especially contemplated expression systems for use herein include various viral transfection systems, and most preferably adenoviral or other pharmaceutically acceptable expression systems such as adeno-associated, lentiviral, and retroviral expression systems. Still further, it is contemplated that suitable alternative expression systems include yeast and artificial chromosome expression systems, and even recombinant expression cassettes that are installed into a host cell's genome using genome editing techniques. However, in especially preferred aspects of the inventive subject matter, the expression system is an adenoviral systems, and especially adenoviral systems with reduced immunogenicity. For example, suitable adenoviral systems include Ad5 with deleted E1 and E2b genes. As most viruses also allow for additional cargo, proteins that can be expressed next to IL-12 include cancer associated antigens, tumor and patient-specific neoepitopes (all of which are preferably HLA matched with respect to the patient and/or directed towards the patient's MHC-I and/or MHC-II presentation pathways). Moreover, further contemplated additional proteins that may be expressed from suitable expression systems include one or more co-stimulatory molecules (e.g., B7.1 (CD80), B7.2 (CD86), ICAM-1 (CD54), ICOS-L, LFA-3 (CD58), 4-1BBL, CD30L, CD40, CD40L, CD48, CD70, CD112, CD155, GITRL, OX40L, TL1A, etc.) and/or one or more checkpoint inhibitors (e.g., protein or peptide that binds to CTLA-4 (CD152) or PD-1 (CD 279)). Additional proteins for expression also include various immune stimulatory cytokines, and particularly IL-2 (especially where IL-2 is retained within the transfected cell as already described above), IL-15, and a IL-15 superagonist (e.g., ALT-803).

With respect to the specific arrangement of sequence elements in the expression systems contemplated herein, it is generally preferred that IL-12 is expressed from a constitutive strong promoter (e.g., SV40, CMV, UBC, EF1A, PGK, CAGG promoter), but inducible promoters are also deemed suitable for use herein, particularly where induction conditions are typical for the tumor microenvironment. For example, inducible promoters include those sensitive to hypoxia and promoters that are sensitive to TGF-β or IL-8 (e.g., via TRAF, JNK, Erk, or other responsive elements promoter). In other examples, suitable inducible promoters include the tetracycline-inducible promoter, the myxovirus resistance 1 (Mx1) promoter, etc. Where additional elements for expression are present, they may be co-expressed from the same promoter and so generate a single transcript, for example, with an internal ribosome entry (IRES) site, or may be transcribed from one or more separate promoters as single gene transcript, as tandem minigenes, or any other arrangement suitable for expression.

In still further contemplated aspects, and especially where viral expression systems are contemplated, it is generally preferred that the viral system is replication deficient and that the host cells have a suitable receptor for entry. For example, where the virus is an adenovirus or a coxsackie virus, the receptor is typically a CAR receptor, which may be natively present or may be expressed in the host cell from a recombinant nucleic acid. On the other hand, where the recombinant nucleic acid for expression of the IL-12 is a linear or circular ‘naked’ nucleic acid (e.g., DNA plasmid or RNA), conventional transfection is typically preferred (e.g., lipofection, electroporation, sonoporation, ballistic transfer, etc.)

Administration of recombinant viruses to the patient, allogenic cells, or patient cells is typically in an amount that will lead to detectable IL-12 in serum, with preferred quantities being typically in the range of 10 to 1000 pg/ml. For example, suitable detectable serum concentrations will be at least 10 pg/ml, at least 25 pg/ml, at least 50 pg/ml, at least 100 pg/ml, or at least 250 pg/ml. However, the exact quantity will generally depend on the MOI, number of infected cells, the strength of promoter, etc. Thus, it should be recognized that a particular serum concentration can be achieved by suitable choice and/or quantity of viral particles, number of infected cells, choice of promoter, etc. For example, where the virus is Ad5 and the promoter is a CMV promoter, typically at least 10⁸, more typically at least 10⁹, even more typically 10¹⁰, and most typically 10¹¹ viral particles will be administered, at least once, and more typically twice, or as often as needed for a therapeutic effect. However, it is generally contemplated that administration is a co-administration such that the IL-2 stimulated NK cells are present at the same time as the expressed or otherwise administered IL-12. For example, IL-2 stimulated NK-92 derivatives (e.g., such as aNK cells, haNK cells, and taNK cells) can be co-administered with IL-12, a IL-12 encoding virus, or an IL-12 antibody conjugate, to thereby increase IFNγ secretion from the stimulated cell as well we increase NKD2D expression in such cells. Additionally, the treatments contemplated herein may also include (metronomic) low dose chemotherapy/radiation to further induce expression of NKG2D ligands on the tumor tissue.

Therefore, in yet another aspect of the inventive subject matter, thusly stimulated NK cells may be used in a pharmaceutical composition, typically formulated as a sterile injectable composition with between 10⁴-10¹¹ cells, and more typically 10⁵-10⁹ cells per dosage unit. Where desirable, these cells may be irradiated at a suitable radiation dosage to prevent further propagation after administration. However, alternative formulations are also deemed suitable for use herein, and all known routes and modes of administration are contemplated herein. As used herein, the term “administering” a pharmaceutical composition or drug refers to both direct and indirect administration of the pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection into the tumor, infusion, oral delivery, topical delivery, etc.).

Therefore, the inventors especially contemplate a method of treating cancer in which constitutively stimulated natural killer cells and IL-12 are co-administered, wherein the IL-12 is administered at a dosage that increases NKG2D expression on the NK cell. Most typically, and as described above, it is generally preferred that the IL-12 is co-administered via expression of a (host or allogenic) cell that is transfected with a recombinant nucleic acid encoding the IL-12 as is exemplarily described below. Likewise, further especially preferred methods include those that increase activity of NK cells or CD8⁺ T-cells in a mammal. In such methods, the mammal is infected with a plurality of recombinant viral particles, each comprising a recombinant nucleic acid segment encoding IL-12 operably coupled to a promoter sequence that drives expression of IL-12 in a cell infected with the recombinant viral particles. Most typically, the number of recombinant viral particles is sufficient to cause expression of a quantity of IL-12 in the infected cells that increases expression of NKG2D on the NK cells or CD8⁺ T-cells in the mammal infected with the virus.

As contemplated methods are thought to increase NKD2D surface expression on various cells in addition to IFNγ secretion, and especially immune competent cells such as NK cells, CD8⁺, and CD4⁺ T-cells, it is further contemplated that treatments may also include one or more steps that increase NKG2D ligand expression and presentation. For example, preferred treatments include low dose chemotherapy and/or low dose radiation therapy, typically performed at dosages that are equal or less than 50%, equal or less than 30%, equal or less than 20%, or equal or less than 10% of the maximum tolerated dose. Moreover, such low dose treatment will preferably be performed in a metronomic fashion, for example, on alternating days, or every third day, or once weekly for several weeks, etc.

### Examples

IFN-γ Production in Response to IL-12 Stimulation:

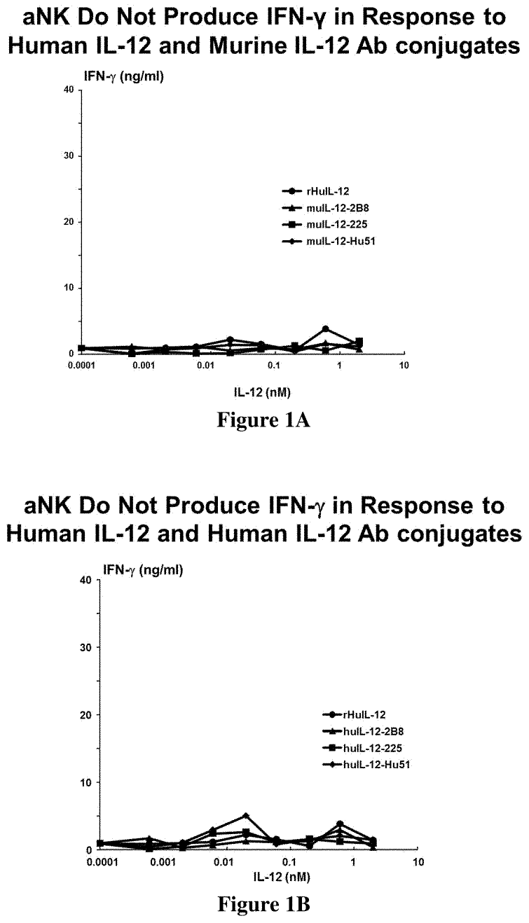

aNK cells without constitutive IL-2 exposure were cultured overnight with human recombinant IL-12, and selected murine recombinant IL-12-Ab conjugates (FIG. 1A), or human recombinant IL-12, and selected human recombinant IL-12-Ab conjugates (FIG. 1B). Cells were cultured by seeding 2.5×10⁵ cells into a 24 well plate, X-Vivo 10 containing 5% human serum. The cell culture supernatants were then collected and human IFN-γ measured by ELISA. As can be readily taken from the graphs in FIGS. 1A and 1B, exposure to IL-12 in various forms did not result in any significant IFNγ secretion.

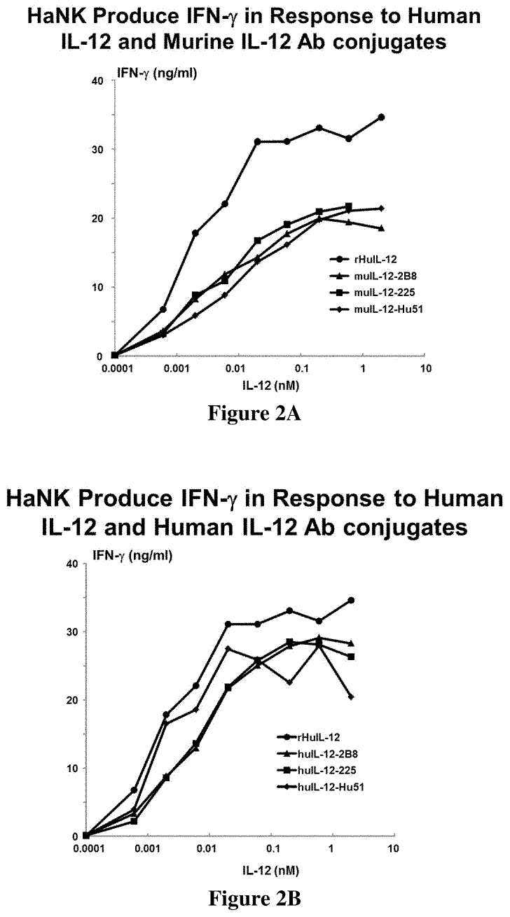

In a further set of experiments, haNK cells (i.e., aNK cell derivatives expressing high-affinity CD16 and intracellularly retained IL-2) were cultured overnight with human recombinant IL-12, and selected murine recombinant IL-12-Ab conjugates (FIG. 2A), or human recombinant IL-12, and selected human recombinant IL-12-Ab conjugates (FIG. 2B). Cells were cultured by seeding 2.5×10⁵ cells into a 24 well plate, X-Vivo 10 containing 5% human serum. The cell culture supernatants were then collected and human IFN-γ measured by ELISA. Remarkably, constitutive IL-2 exposure via intracellular expression of IL-2 rendered aNK cells sensitive to IL-12 signaling as can be readily taken from the graphs in FIGS. 2A and 2B. Indeed, exposure to IL-12 in various forms did result in significant IFNγ secretion for both murine and human IL-12. As expected, human IL-12 produced somewhat stronger IFNγ secretion in the haNK cells than murine IL-12.

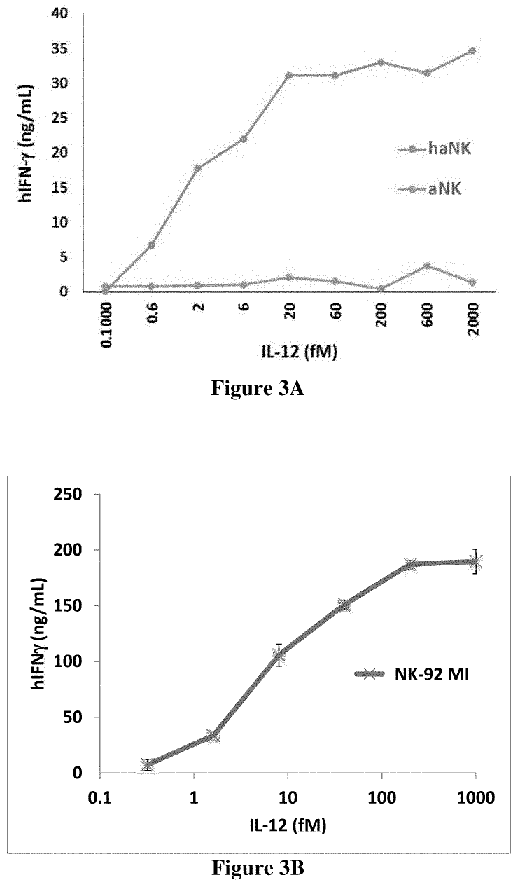

FIG. 3A comparatively depicts the data for aNK cells without constitutive IL-2 exposure and haNK cells with constitutive IL-2 exposure. In yet another experiment, the inventors modified aNK cells by stable integration of an IL-2 expression cassette (to so form NK-92MI cells not expressing the high affinity CD16 variant). As can be taken from the results in FIG. 3B, such modified NK cells were not only responsive to IL-12 signaling to secrete IFNγ, but secreted unexpected high quantities of IFNγ (peaking near 200 ng/ml). NK92-MI production: NK-92 cells were transfected with human IL-2 cDNA in a retroviral MFG-hIL-2 vector by particle-mediated gene transfer. The transfection was stable. NK-92 and this derivative cell line NK-92MI had the following characteristics: surface marker positive for CD2, CD7, CD11a, CD28, CD45, CD54 and CD56 bright; surface marker negative for CD1, CD3, CD4, CD5, CD8, CD10, CD14, CD16, CD19, CD20, CD23, CD34 and HLA-DR.

Expression of IL-12 in the Ad5 [E1-, E2b-] Vector:

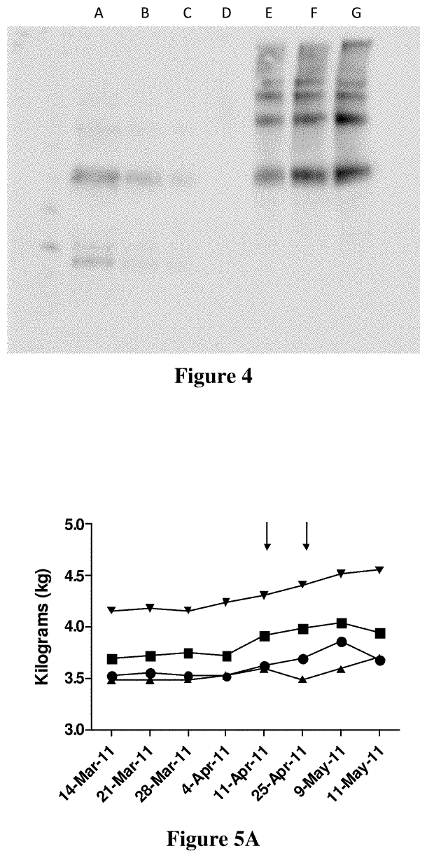

The gene for human IL-12 was inserted into the Ad5 [E1-, E2b-] viral vector backbone (e.g., J Virol. 1998 February; 72(2):926-33). The expression of IL-12 was confirmed by infecting human cells (A549) with the Ad5 [E1-, E2b-]-IL-12 recombinant virus and IL-12 production was confirmed by Western Blot analysis as can be seen in FIG. 4 where expression of IL-12 in human cells infected with Ad5 [E1-, E2b-]-IL-12 is shown. More particularly, A549 cells were infected at an MOI of 1000 with Ad5 [E1-, E2b-]-IL-12, and IL-12 expression was confirmed by western blot analysis. Recombinant IL-12 was used as a positive control and uninfected A549 cells served as a negative control. The samples are visualized in FIG. 4 in the following order: A. 100 ng IL-12 reference material, B. 50 ng IL-12 reference material, C. 25 ng IL-12 reference material, D. Negative, E. Ad5 [E1-, E2b-]-IL-12 lysate (10 uL), F. Ad5 [E1-, E2b-]-IL-12 lysate (17 uL), G. Ad5 [E1-, E2b-]-IL-12 lysate (25 uL).

In Vivo Administration of Ad5 [E1-, E2b-]-IL-12:

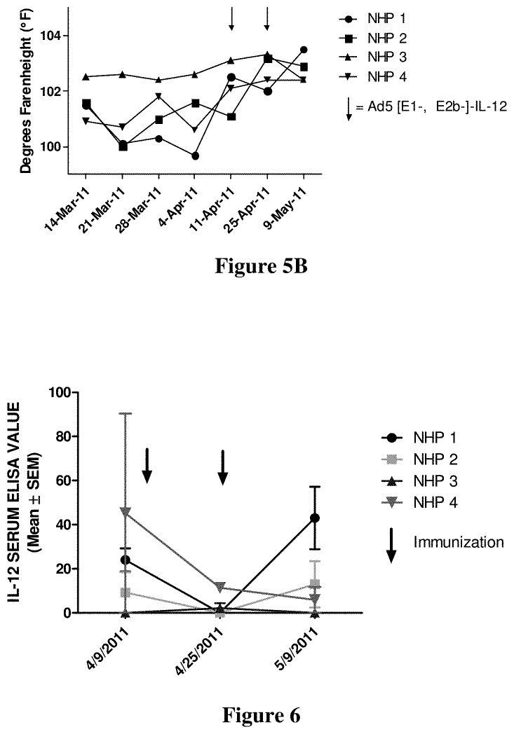

Four non-human primates (NHP) were immunized in the hind leg with 1×10¹¹ VP of Ad5 [E1-, E2b-]-IL-12 and 4×10¹¹ VP of Ad5 [E1-, E2b-]-gag/pol/nef/env (5×10¹¹ VP) twice at a two-week interval. Assuming a human weight of 60 kg and the average weight of the NHP was 3.9 kg, a 5×10¹¹ VP/dose in these NHP is the NHP-to-human equivalent of 7.7×10¹² VP/dose in humans. To monitor for adverse effects from the treatment, clinical observations were recorded including the animal's weights and temperatures. The animals did not experience any weight loss as is shown in FIG. 5A or become febrile as can be seen in FIG. 5B after administration of Ad5 [E1-, E2b-]-IL-12 and Ad5 [E1-, E2b-]-gag/pol/nef/env. Here, each line represents an individual animal. Arrow indicate days that the animals were administered the Ad5 [E1-, E2b-] treatments. Also, the inguinal and axillary lymphnodes were examined and they remained normal for the course of the study. These data indicate that Ad5 [E1-, E2b-]-IL-12 can be given concurrently with other vectored transgenes even at very high doses without adverse effects. Serum levels of IL-12 were determined in the NHP treated with Ad5 [E1-, E2b-]-IL-12 by quantitative ELISA and exemplary results are shown in FIG. 6. More particularly, four rhesus macaques were administered 1×10¹¹ VP of Ad5 [E1-, E2b-]-IL-12 and 4×10¹¹ VP of Ad5 [E1-, E2b-]-gag/pol/nef/env (5×10¹¹ VP) twice at a two week interval in the hind leg. The level of IL-12 in serum was determined by a quantitative ELISA. Dates refer to when samples were tested.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Moreover, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

1. A method of stimulating a genetically modified NK cell, comprising:
exposing a genetically modified NK cell constitutively to IL-2 to thereby sensitize the genetically modified NK cell to IL-12; and
exposing the sensitized cell to IL-12 to stimulate interferon gamma (IFNγ) secretion by the sensitized cell.

2. (canceled)

3. The method of claim 1 wherein the genetically modified NK cell is an aNK cell.

4. The method of claim 1 wherein the genetically modified NK cell is constitutively exposed to at least 100 IU/ml IL-2.

5. The method of claim 1 wherein the IL-2 is a pegylated IL-2.

6. The method of claim 1 wherein the genetically modified NK cell is constitutively exposed to IL-2 by intracellular expression of IL-2.

7. The method of claim 6 wherein the genetically modified NK cell is a haNK cell.

8. The method of claim 1 wherein the IL-12 is expressed from a cell that is infected with a recombinant virus, and wherein the recombinant virus includes a sequence segment that encodes the IL-12.

9. The method of claim 8 wherein the recombinant virus includes a second sequence segment that encodes at least one of a tumor and patient specific antigen, a tumor associated antigen, and a tumor specific antigen.

10. The method of claim 1 wherein the IL-12 is coupled to an antibody.

11. The method of claim 10 wherein the antibody binds to a cancer cell.

12. The method of claim 1 wherein the genetically modified NK cell is exposed to the IL-2 in vitro, and wherein the sensitized cell is administered to a patient.

13. The method of claim 1 wherein the sensitized cell is exposed to the IL-12 in vitro, and wherein the sensitized cell is administered to a patient.

14. A method of treating cancer, comprising:
administering a sensitized genetically modified NK cell to an individual diagnosed with cancer, wherein the genetically modified NK cell is sensitized by constitutive exposure to IL-2; and
administering an IL-12 antibody conjugate or a recombinant virus to the individual that encodes IL-12 to stimulate interferon gamma (IFNγ) secretion by the sensitized genetically modified NK cell.

15. (canceled)

16. The method of claim 14 wherein the genetically modified NK cell is an aNK cell.

17. The method of claim 14 wherein the genetically modified NK cell is constitutively exposed to at least 100 IU/ml IL-2.

18. The method of claim 14 wherein the IL-2 is a pegylated IL-2.

19. The method of claim 14 wherein the genetically modified NK cell is constitutively exposed to IL-2 by intracellular expression of IL-2.

20. The method of claim 19 wherein the genetically modified NK cell is a haNK cell.

21-25. (canceled)

26. A sensitized genetically modified NK cell produced by the method of claim 1.

27-34. (canceled)

35. A method of increasing activity of NK cells or CDS+ T-cells in a mammal, comprising:
infecting cells of the mammal with a plurality of recombinant viral particles, each viral particle comprising a recombinant nucleic acid segment encoding IL-12 operably coupled to a promoter sequence that drives expression of IL-12 in a cell infected with the recombinant viral particles;
wherein the plurality of recombinant viral particles is sufficient to cause expression of a quantity of IL-12 in the infected cells that increases expression of NKG2D on the NK cells or CDS+ T-cells in the mammal infected with the virus.

36-41. (canceled)
